(19) United States
(12) Patent Application Publication
(10) Pub. No.: US 2019/0183901 A1
(43) Pub. Date: Jun. 20, 2019

(54) COMBINATIONS AND USES AND TREATMENTS THEREOF

(71) Applicant: ViiV HEALTHCARE COMPANY, Wilmington, DE (US)

(72) Inventor: Brian Alvin JOHNS, Research Triangle Park, NC (US)

(21) Appl. No.: 16/326,284

(22) PCT Filed: Aug. 29, 2017
(86) PCT No.: PCT/US2017/049046

§ 371 Date: Feb. 18, 2019

Related U.S. Application Data
(60) Provisional application No. 62/381,789, filed on Aug. 31, 2016.

(51) Int. Cl. A61K 31/537; A61K 31/54; A61K 31/513; A61K 31/427; A61P 31/18

(52) U.S. Cl. CPC A61K 31/537; A61K 31/54; A61P 31/18; A61K 31/427; A61K 31/513

(57) ABSTRACT

Methods for treating HIV in a human using combinations of: bictegravir and one of two different maturation inhibitors are disclosed, as well as compositions containing such compounds are provided.

### FIELD OF THE INVENTION

Methods for treating HIV in a human using a combination of: bictegravir and at least one of two different maturation inhibitors are disclosed, as well as compositions comprising such compounds.

### BACKGROUND OF THE INVENTION

Human immunodeficiency virus infection and related diseases are a major public health problem worldwide. Human immunodeficiency virus type 1 (HIV-1) encodes three enzymes that are required for viral replication: reverse transcriptase, protease, and integrase. Although drugs targeting reverse transcriptase and protease are in wide use and have shown effectiveness, particularly when employed in combination, toxicity and development of resistant strains have limited their usefulness (Palella, et al., N. Engl. J. Med. (1998) 338:853-860; Richman, Nature (2001) 410:995-1001).

A goal of antiretroviral therapy is to achieve viral suppression in the HIV-infected human. Treatment guidelines published by the United States Department of Health and Human Services provide that achievement of viral suppression requires the use of combination therapies, i.e., several drugs from at least two or more drug classes. (PANEL ON ANTIRETROVIRAL GUIDELINES FOR ADULTS AND ADOLESCENTS: GUIDELINES FOR THE USE OF ANTIRETROVIRAL AGENTS IN HIV-1-INFECTED ADULTS AND ADOLESCENTS. Department of Health and Human Services. Available at http://aidsinfo.nih.gov/ContentFiles/AdultandAdolescentGL.pdf. Section accessed Mar. 14, 2013.) In addition, decisions regarding the treatment of HIV-infected humans are complicated when the human requires treatment for other medical conditions. Id. at E-12. Because the standard of care requires the use of multiple different drugs to suppress HIV, as well as to treat other conditions the human may be experiencing, the potential for drug interaction is a criterion for selection of a drug regimen. As such, there is a need for antiretroviral therapies having a decreased potential for drug interactions and with even more therapeutic potencies.

Although different combinations of antiretroviral drugs have been developed for the treatment of HIV, a need still exists for further HIV treatment regimens for even more potencies or ease of use.

### SUMMARY OF THE INVENTION

The present inventors have identified a combination of therapeutic agents, uses thereof, and associated methods of treatment with advantages over previously known agents and combinations.

In one embodiment, this invention provides a combination comprising a compound of bictegravir (formula I):

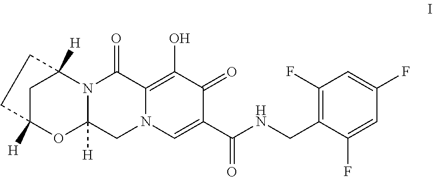 [SMILES: O=C(NCc1c(F)cc(F)cc1F)c1cn2c(c(O)c1=O)C(=O)N1[C@H]3CC[C@H](C3)O[C@@H]1C2]

or a pharmaceutically acceptable salt thereof; and a compound of formula II, a maturation inhibitor:

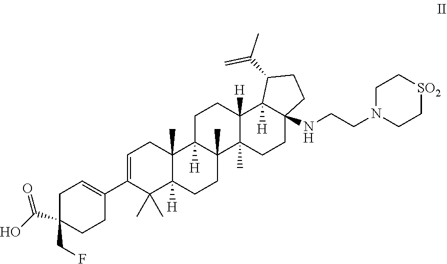

or a pharmaceutically acceptable salt thereof;

In another embodiment, this invention provides a pharmaceutical composition comprising the combination of bictegravir, or a pharmaceutically acceptable salt thereof, and the compound of formula II, or a pharmaceutically acceptable salt thereof, in association with one or more therapeutically acceptable carriers therefor.

In an additional embodiment, this invention provides a combination of bictegravir, or a pharmaceutically acceptable salt thereof, and the compound of formula II, a pharmaceutically acceptable salt thereof, for use in medical therapy.

A further embodiment of this invention provides a method for treating human immunodeficiency virus (HIV) in a human, comprising administering to the human a therapeutically effective amount of bictegravir, or a pharmaceutically acceptable salt thereof; and a therapeutically effective amount of a compound of formula II, or a pharmaceutically acceptable salt thereof.

In yet another embodiment, bictegravir, or a pharmaceutically acceptable salt thereof, and the compound of formula II, or a pharmaceutically acceptable salt thereof, are co-administered in separate dosage forms.

In an alternative embodiment, bictegravir, or a pharmaceutically acceptable salt thereof, and the compound of formula II, or a pharmaceutically acceptable salt thereof, are co-administered in a single dosage form.

In yet another embodiment, this method further comprises administering an agent chosen from: emtricitabine and lamivudine.

In one embodiment, this invention provides a combination comprising a compound of bictegravir (formula I):

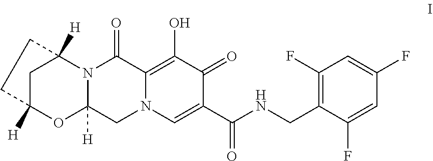 [SMILES: O=C(NCc1c(F)cc(F)cc1F)c1cn2c(c(O)c1=O)C(=O)N1[C@H]3CC[C@H](C3)O[C@@H]1C2]

or a pharmaceutically acceptable salt thereof; and

a compound of formula III, a maturation inhibitor:

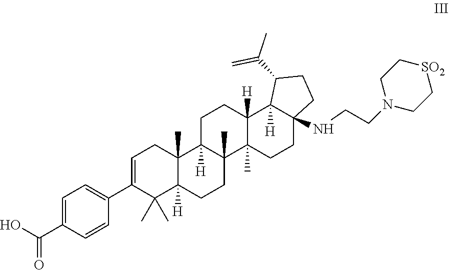

or a pharmaceutically acceptable salt thereof;

In another embodiment, this invention provides a pharmaceutical composition comprising the combination of bictegravir, or a pharmaceutically acceptable salt thereof, and the compound of formula III, or a pharmaceutically acceptable salt thereof, in association with one or more therapeutically acceptable carriers therefor.

In an additional embodiment, this invention provides a combination of bictegravir, or a pharmaceutically acceptable salt thereof, and the compound of formula III, a pharmaceutically acceptable salt thereof, for use in medical therapy.

A further embodiment of this invention provides a method for treating human immunodeficiency virus (HIV) in a human, comprising administering to the human a therapeutically effective amount of bictegravir, or a pharmaceutically acceptable salt thereof; and a therapeutically effective amount of a compound of formula II, or a pharmaceutically acceptable salt thereof.

In yet another embodiment, bictegravir, or a pharmaceutically acceptable salt thereof, and the compound of formula II, or a pharmaceutically acceptable salt thereof, are co-administered in separate dosage forms.

In an alternative embodiment, bictegravir, or a pharmaceutically acceptable salt thereof, and the compound of formula II, or a pharmaceutically acceptable salt thereof, are co-administered in a single dosage form.

In yet another embodiment, this method further comprises administering an agent chosen from: emtricitabine and lamivudine.

A further embodiment of this invention provides a method for treating human immunodeficiency virus (HIV) in a human, comprising administering to the human a therapeutically effective amount of bictegravir, or a pharmaceutically acceptable salt thereof; and a therapeutically effective amount of a compound of formula III, or a pharmaceutically acceptable salt thereof.

In yet another embodiment, bictegravir, or a pharmaceutically acceptable salt thereof, and the compound of formula III, or a pharmaceutically acceptable salt thereof, are co-administered in separate dosage forms.

In an alternative embodiment, bictegravir, or a pharmaceutically acceptable salt thereof, and the compound of formula III, or a pharmaceutically acceptable salt thereof, are co-administered in a single dosage form.

In yet another embodiment, this method further comprises administering an agent chosen from: emtricitabine and lamivudine.

In another embodiment, this invention provides a pharmaceutical composition comprising the combination of bictegravir, or a pharmaceutically acceptable salt thereof, and the compound of formula II, or a pharmaceutically acceptable salt thereof, in association with one or more therapeutically acceptable carriers therefor, wherein said composition further comprises an agent chosen from emtricitabine and lamivudine.

In yet another embodiment, this invention provides a pharmaceutical composition comprising a therapeutically effective amount of the combination of bictegravir, or a pharmaceutically acceptable salt thereof, and the compound of formula II, or a pharmaceutically acceptable salt thereof, wherein said composition further comprises emtricitabine and ritonavir.

In yet another embodiment, this invention provides a pharmaceutical composition comprising a therapeutically effective amount of the combination of bictegravir, or a pharmaceutically acceptable salt thereof, and the compound of formula II, or a pharmaceutically acceptable salt thereof, wherein said composition further comprises emtricitabine and cobicistat.

In yet another embodiment, this invention provides a pharmaceutical composition comprising a therapeutically effective amount of the combination of bictegravir, or a pharmaceutically acceptable salt thereof, and the compound of formula II, or a pharmaceutically acceptable salt thereof, wherein said composition further comprises lamivudine.

In yet another embodiment, this invention provides a pharmaceutical composition comprising a therapeutically effective amount of the combination of bictegravir, or a pharmaceutically acceptable salt thereof, and the compound of formula II, or a pharmaceutically acceptable salt thereof, wherein said composition further comprises lamivudine and ritonavir.

In yet another embodiment, this invention provides a pharmaceutical composition comprising a therapeutically effective amount of the combination of bictegravir, or a pharmaceutically acceptable salt thereof, and the compound of formula II, or a pharmaceutically acceptable salt thereof, wherein said composition further comprises lamivudine and cobicistat.

In an additional embodiment, this invention provides a combination of bictegravir, or a pharmaceutically acceptable salt thereof, and the compound of formula II, a pharmaceutically acceptable salt thereof, for use in medical therapy.

In an additional embodiment, this invention provides a method for preventing human immunodeficiency virus (HIV) in a human, comprising administering to the human a therapeutically effective amount of bictegravir (formula I):

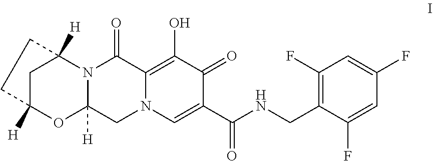 [SMILES: O=C(NCc1c(F)cc(F)cc1F)c1cn2c(c(O)c1=O)C(=O)N1[C@H]3CC[C@H](C3)O[C@@H]1C2]

or a pharmaceutically acceptable salt thereof; and

a therapeutically effective amount of a compound of formula II:

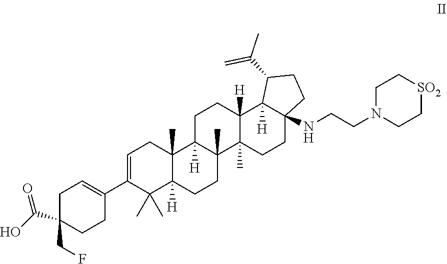

or a pharmaceutically acceptable salt thereof.

In yet another embodiment, bictegravir, or a pharmaceutically acceptable salt thereof, is administered orally to assess its safety and tolerability, and, if no or low issue in safety and tolerability is found (called “oral-lead method”), then bictegravir, or a pharmaceutically acceptable salt thereof, is administered orally, and the compound of formula II, or a pharmaceutically acceptable salt thereof, is administered parenterally.

In an additional embodiment, this invention provides a method for preventing human immunodeficiency virus (HIV) in a human, comprising administering to the human a therapeutically effective amount of bictegravir (formula I):

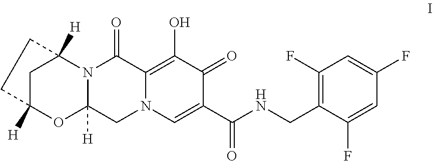 [SMILES: O=C(NCc1c(F)cc(F)cc1F)c1cn2c(c(O)c1=O)C(=O)N1[C@H]3CC[C@H](C3)O[C@@H]1C2]

or a pharmaceutically acceptable salt thereof; and

a therapeutically effective amount of a compound of formula III:

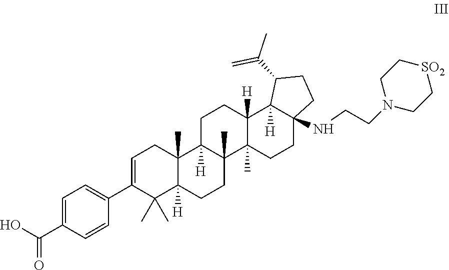

or a pharmaceutically acceptable salt thereof.

In yet another embodiment, bictegravir, or a pharmaceutically acceptable salt thereof, is administered orally to assess its safety and tolerability, and, if no or low issue in safety and tolerability is found (called “oral-lead method”), then bictegravir, or a pharmaceutically acceptable salt thereof, is administered orally, but the compound of formula III, or a pharmaceutically acceptable salt thereof, is administered parenterally.

In another embodiment, this invention provides a pharmaceutical composition comprising a therapeutically effective amount of bictegravir (formula I):

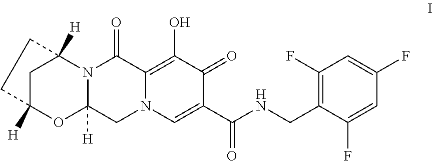 [SMILES: O=C(NCc1c(F)cc(F)cc1F)c1cn2c(c(O)c1=O)C(=O)N1[C@H]3CC[C@H](C3)O[C@@H]1C2]

or a pharmaceutically acceptable salt thereof; and

a therapeutically effective amount of a compound of formula II:

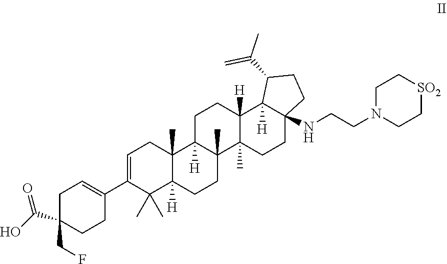

or a pharmaceutically acceptable salt thereof, in combination with at least one pharmaceutically acceptable carrier, and, optionally, ritonavir.

In yet another embodiment, this invention provides a pharmaceutical composition comprising a therapeutically effective amount of the combination of bictegravir, or a pharmaceutically acceptable salt thereof, and the compound of formula II, or a pharmaceutically acceptable salt thereof, wherein said composition further an agent chosen from: emtricitabine and lamivudine.

In another embodiment, this invention provides a pharmaceutical composition comprising a therapeutically effective amount of bictegravir (formula I):

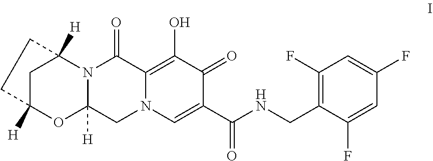 [SMILES: O=C(NCc1c(F)cc(F)cc1F)c1cn2c(c(O)c1=O)C(=O)N1[C@H]3CC[C@H](C3)O[C@@H]1C2]

or a pharmaceutically acceptable salt thereof; and

a therapeutically effective amount of a compound of formula III:

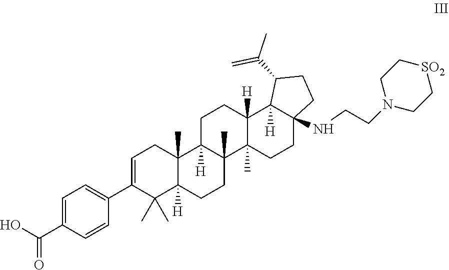

or a pharmaceutically acceptable salt thereof, in combination with at least one pharmaceutically acceptable carrier, and, optionally, ritonavir.

In yet another embodiment, this invention provides a pharmaceutical composition comprising a therapeutically effective amount of the combination of bictegravir, or a pharmaceutically acceptable salt thereof, and the compound of formula II, or a pharmaceutically acceptable salt thereof, wherein said composition further comprises an agent chosen from: emtricitabine and lamivudine.

In yet another embodiment, this invention provides a pharmaceutical composition comprising a therapeutically effective amount of the combination of bictegravir, or a pharmaceutically acceptable salt thereof, and the compound of formula III, or a pharmaceutically acceptable salt thereof, wherein said composition further comprises emtricitabine and ritonavir

In yet another embodiment, this invention provides a pharmaceutical composition comprising a therapeutically effective amount of the combination of bictegravir, or a pharmaceutically acceptable salt thereof, and the compound of formula III, or a pharmaceutically acceptable salt thereof, wherein said composition further comprises emtricitabine and cobicistat

In yet another embodiment, this invention provides a pharmaceutical composition comprising a therapeutically effective amount of the combination of bictegravir, or a pharmaceutically acceptable salt thereof, and the compound of formula III, or a pharmaceutically acceptable salt thereof, wherein said composition further comprises lamivudine.

In yet another embodiment, this invention provides a pharmaceutical composition comprising a therapeutically effective amount of the combination of bictegravir, or a pharmaceutically acceptable salt thereof, and the compound of formula III, or a pharmaceutically acceptable salt thereof, wherein said composition further comprises lamivudine and ritonavir.

In yet another embodiment, this invention provides a pharmaceutical composition comprising a therapeutically effective amount of the combination of bictegravir, or a pharmaceutically acceptable salt thereof, and the compound of formula III, or a pharmaceutically acceptable salt thereof, wherein said composition further comprises lamivudine and cobicistat.

In an alternative embodiment, this invention provides a kit comprising:
(1) a composition comprising bictegravir, or a pharmaceutically acceptable salt thereof;
(2) a composition comprising the compound of formula II, or a pharmaceutically acceptable salt thereof; and
(3) instructions for their co-administration.

In yet another embodiment, this invention provides a kit comprising:
(1) a composition comprising bictegravir, or a pharmaceutically acceptable salt thereof;
(2) a composition comprising the compound of formula III, or a pharmaceutically acceptable salt thereof; and
(3) instructions for their co-administration.

### DETAILED DESCRIPTION OF THE INVENTION

U.S. Pat. No. 9,216,996 describes a class of HIV integrase inhibitors of (2R,5S,13aR)-8-hydroxy-7,9-dioxo-N-[(2,4,6-trifluorophenyl)methyl]-2,3,4,5,7,9,13,13a-octahydro-2,5-methanopyrido[1′,2′:4,5]pyrazino[2,1-b][1,3]oxazepine-10-carboxamide, which is currently being developed for the treatment and prevention of HIV. Among them is an agent called bictegravir. Bictegravir has the structure of formula I below:

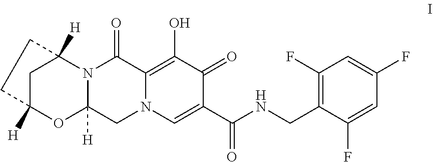 [SMILES: O=C(NCc1c(F)cc(F)cc1F)c1cn2c(c(O)c1=O)C(=O)N1[C@H]3CC[C@H](C3)O[C@@H]1C2]

Methods for preparing bictegravir are described in U.S. Pat. No. 9,216,996.

Certain derivatives of betulinic acid have now been shown to exhibit potent anti-HIV activity as HIV maturation inhibitors. Maturation inhibitors an emerging class of compounds for the treatment of HIV are called HIV maturation inhibitors (MI). In particular, reference is made herein to an this US patent discloses one such MI inhibitor that is a potential candidate for the treatment and prevention of HIV, and has the structure of formula II below:

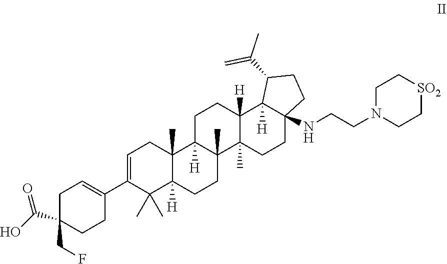

The compound of formula II is (1R)-4-[(1R,3aS,5aR,5bR,7aR,11aS,11bR,13aR,13bR)-3a-{[2-(1,1-dioxido-4-thiomorpholinyl)ethyl]amino}-5a,5b,8,8,11a-pentamethyl-1-(1-propen-2-yl)-2,3,3a,4,5,5a,5b,6,7,7a,8,11,11a,11b,12,13,13a,13b-octadecahydro-1H-cyclopenta[a]chrysen-9-yl]-1-(fluoromethyl)-3-cyclohexene-1-carboxylic acid methods of preparing the compound of formula II are described in WO 2015/157483.

Another such MI inhibitor, 4-((1R,3aS,5aR,5bR,7aR,11aS,11bR,13aR,13bR)-3a-((2-(1,1-dioxido-4-thiomorpholinyl)ethyl)amino)-1-isopropenyl-5a,5b,8,8,11a-pentamethyl-2,3,3a,4,5,5a,5b,6,7,7a,8,11,11a,11b,12,13,13a,13b-octadecahydro-1Hcyclopenta[a]chrysen-9-yl)benzoic acid, which is currently undergoing clinical trials for the treatment and prevention of HIV, has the structure of formula III:

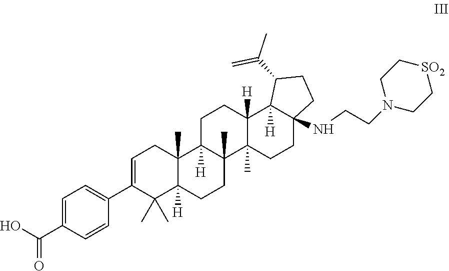

Methods of preparing the compound of formula III are described in U.S. Pat. No. 8,846,647.

Bictegravir and/or the compound of formula II or formula III may contain one or more chiral atoms, or may cyclopenta[a]chrysen-9-yl)oxy)-2,2-dimethyl-4-oxobutanoic acid. Methods of preparing the compound otherwise be capable of existing as enantiomers. Accordingly, the compounds of this invention include mixtures of enantiomers, as well as purified enantiomers or enantiomerically enriched mixtures. Also, it is understood that all tautomers and mixtures of tautomers are included within the scope of bictegravir and the compound of formula II or formula III.

One embodiment of the invention provides a method for treating HIV infection in a human comprising administering to the human a therapeutically effective amount of the compound of formula I (bictegravir):

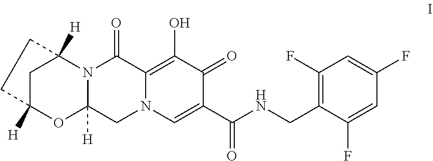 [SMILES: O=C(NCc1c(F)cc(F)cc1F)c1cn2c(c(O)c1=O)C(=O)N1[C@H]3CC[C@H](C3)O[C@@H]1C2]

or a pharmaceutically acceptable salt thereof; and a therapeutically effective amount of a compound of formula II:

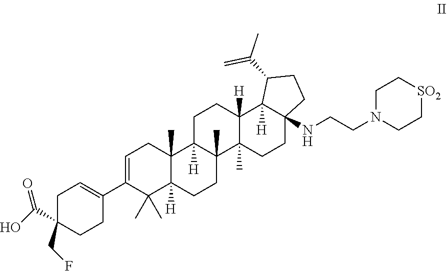

or a pharmaceutically acceptable salt thereof.

Another embodiment of the invention provides a method for treating HIV infection in a human comprising administering to the human a therapeutically effective amount of the compound of formula I (bictegravir):

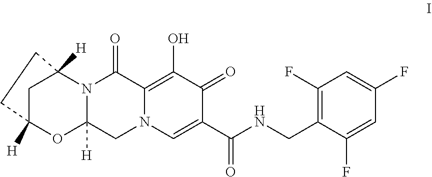 [SMILES: O=C(NCc1c(F)cc(F)cc1F)c1cn2c(c(O)c1=O)C(=O)N1[C@H]3CC[C@H](C3)O[C@@H]1C2]

or a pharmaceutically acceptable salt thereof; and a therapeutically effective amount of a compound of formula III:

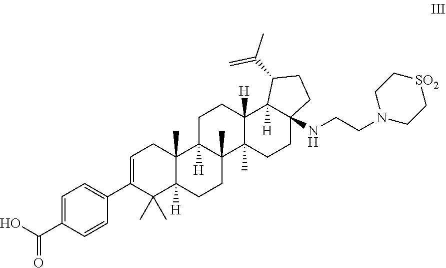

or a pharmaceutically acceptable salt thereof.

Another embodiment of the invention provides a method for preventing HIV infection in a human comprising administering to the human a therapeutically effective amount of the compound of formula I (bictegravir):

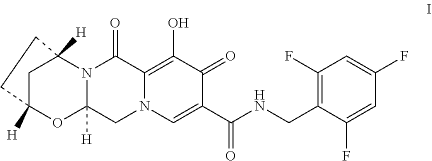 [SMILES: O=C(NCc1c(F)cc(F)cc1F)c1cn2c(c(O)c1=O)C(=O)N1[C@H]3CC[C@H](C3)O[C@@H]1C2]

or a pharmaceutically acceptable salt thereof; and

a therapeutically effective amount of a compound of formula II:

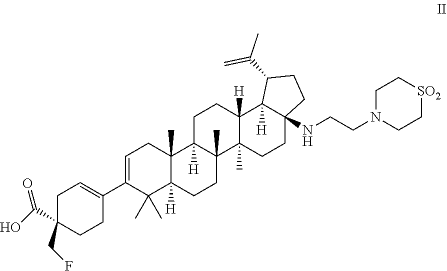

or a pharmaceutically acceptable salt thereof.

In another embodiment, bictegravir, or a pharmaceutically acceptable salt thereof, and the compound of formula II, or a pharmaceutically acceptable salt thereof, are co-administered in separate dosage forms.

In another embodiment, bictegravir or a pharmaceutically acceptable salt thereof, and the compound of formula II, or a pharmaceutically acceptable salt thereof, are co-administered in a single dosage form.

In yet another embodiment, bictegravir, or a pharmaceutically acceptable salt thereof, is administered orally to assess its safety and tolerability, and, if no or low issue in safety and tolerability is found (called “oral-lead method”), then bictegravir, or a pharmaceutically acceptable salt thereof, is administered intramuscularly or subcutaneously, but the compound of formula II, or a pharmaceutically acceptable salt thereof, is orally administered.

In yet another embodiment, bictegravir, or a pharmaceutically acceptable salt thereof, is administered orally to assess its safety and tolerability, and, if no or low issue in safety and tolerability is found (called “oral-lead method”), then bictegravir, or a pharmaceutically acceptable salt thereof, is administered orally, but the compound of formula II, or a pharmaceutically acceptable salt thereof, is administered parenterally.

In yet another embodiment, bictegravir, or a pharmaceutically acceptable salt thereof, is administered orally, and the compound of formula II, or a pharmaceutically acceptable salt thereof, is administered parenterally. In one embodiment, the compound of formula II is administered in a long acting and/or implantable formulation.

In a further embodiment, bictegravir, or a pharmaceutically acceptable salt thereof, and the compound of formula II, or a pharmaceutically acceptable salt thereof, are both administered orally.

In yet another embodiment, bictegravir, or a pharmaceutically acceptable salt thereof, is administered orally, and the compound of formula II, or a pharmaceutically acceptable salt thereof, is administered parenterally.

In an additional embodiment, both bictegravir, or a pharmaceutically acceptable salt thereof, and the compound of formula II, or a pharmaceutically acceptable salt thereof, are administered parenterally, either together, or separately. In one embodiment, the parenteral administration is a long acting parenteral.

In yet another embodiment, both bictegravir, or a pharmaceutically acceptable salt thereof, and the compound of formula II, or a pharmaceutically acceptable salt thereof, are both administered parenterally.

In yet another embodiment, bictegravir, or a pharmaceutically acceptable salt thereof, is administered orally to assess its safety and tolerability, and, if no or low issue in safety and tolerability is found (called “oral-lead method”), then bictegravir, or a pharmaceutically acceptable salt thereof, is administered intramuscularly or subcutaneously, but the compound of formula II, or a pharmaceutically acceptable salt thereof, is orally administered.

In yet another embodiment, bictegravir, or a pharmaceutically acceptable salt thereof, is administered orally to assess its safety and tolerability, and, if no or low issue in safety and tolerability is found (called “oral-lead method”), then bictegravir, or a pharmaceutically acceptable salt thereof, is administered orally, but the compound of formula II, or a pharmaceutically acceptable salt thereof, is administered parenterally.

Another embodiment of the invention provides a method for preventing HIV infection in a human comprising administering to the human a therapeutically effective amount of the compound of formula I (bictegravir):

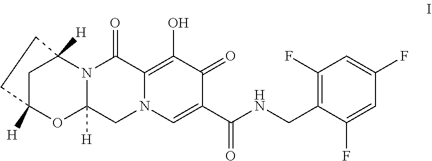 [SMILES: O=C(NCc1c(F)cc(F)cc1F)c1cn2c(c(O)c1=O)C(=O)N1[C@H]3CC[C@H](C3)O[C@@H]1C2]

or a pharmaceutically acceptable salt thereof; and

a therapeutically effective amount of a compound of formula III:

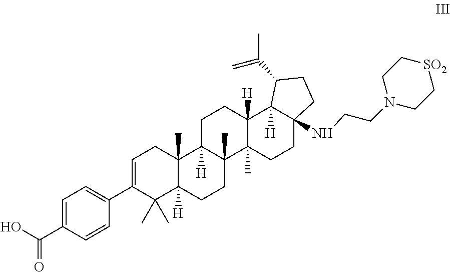

or a pharmaceutically acceptable salt thereof.

In another embodiment, bictegravir, or a pharmaceutically acceptable salt thereof, and the compound of formula III, or a pharmaceutically acceptable salt thereof, are co-administered in separate dosage forms.

In another embodiment, bictegravir or a pharmaceutically acceptable salt thereof, and the compound of formula III, or a pharmaceutically acceptable salt thereof, are co-administered in a single dosage form.

In yet another embodiment, bictegravir, or a pharmaceutically acceptable salt thereof, is administered parenterally, and the compound of formula III, or a pharmaceutically acceptable salt thereof, is administered orally. In a further embodiment, both bictegravir, or a pharmaceutically acceptable salt thereof, and the compound of formula III, or a pharmaceutically acceptable salt thereof, are administered parenterally.

In a further embodiment, bictegravir, or a pharmaceutically acceptable salt thereof, and the compound of formula III, or a pharmaceutically acceptable salt thereof, are both administered orally.

In yet another embodiment, bictegravir, or a pharmaceutically acceptable salt thereof, is administered orally to assess its safety and tolerability, and, if no or low issue in safety and tolerability is found (called “oral-lead method”), then bictegravir, or a pharmaceutically acceptable salt thereof, is administered intramuscularly or subcutaneously, but the compound of formula III, or a pharmaceutically acceptable salt thereof, is orally administered.

In yet another embodiment, bictegravir, or a pharmaceutically acceptable salt thereof, is administered orally to assess its safety and tolerability, and, if no or low issue in safety and tolerability is found (called “oral-lead method”), then bictegravir, or a pharmaceutically acceptable salt thereof, is administered orally, but the compound of formula III, or a pharmaceutically acceptable salt thereof, is administered parenterally.

In yet another embodiment, bictegravir, or a pharmaceutically acceptable salt thereof, is administered orally, and the compound of formula III, or a pharmaceutically acceptable salt thereof, is administered parenterally. In one embodiment, the compound of formula III is administered in a long acting parenteral and/or implantable formulation.

In an additional embodiment, both bictegravir, or a pharmaceutically acceptable salt thereof, and the compound of formula III, or a pharmaceutically acceptable salt thereof, are administered parenterally, either together, or separately. In one embodiment, the parenteral administration is a long acting parenteral.

In yet another embodiment, both bictegravir, or a pharmaceutically acceptable salt thereof, and the compound of formula III, or a pharmaceutically acceptable salt thereof, are both administered parenterally. In one embodiment, pharmaceutical compositions comprising bictegravir, or a pharmaceutically acceptable salt thereof, and the compound of formula II, or a pharmaceutically acceptable salt thereof, and a pharmaceutically acceptable carrier, diluent or excipient are provided.

In one embodiment, pharmaceutical compositions consisting essentially of bictegravir or a pharmaceutically acceptable salt thereof, and the compound of formula II, or a pharmaceutically acceptable salt thereof, and a pharmaceutically acceptable carrier, diluent or excipient are provided.

In one embodiment, pharmaceutical compositions consisting of bictegravir, or a pharmaceutically acceptable salt thereof, and the compound of formula II, or a pharmaceutically acceptable salt thereof, and one or more pharmaceutically acceptable carriers, diluents or excipients are provided.

In yet another embodiment, combinations comprising bictegravir, or a pharmaceutically acceptable salt thereof, and a compound of formula II, or a pharmaceutically acceptable salt thereof, are provided.

In yet another embodiment, combinations consisting essentially of bictegravir, or a pharmaceutically acceptable salt thereof; and a compound of formula II, or a pharmaceutically acceptable salt thereof, are provided.

In yet another embodiment, combinations consisting of bictegravir, or a pharmaceutically acceptable salt thereof; and a compound of formula II, or a pharmaceutically acceptable salt thereof; and one or more pharmaceutically acceptable carriers, diluents or excipients are provided.

Another embodiment provides further administration of at least one antiretroviral compound.

In another embodiment, pharmaceutical compositions comprising bictegravir, or a pharmaceutically acceptable salt thereof, and the compound of formula III, or a pharmaceutically acceptable salt thereof, and a pharmaceutically acceptable carrier, diluent or excipient are provided.

In one embodiment, pharmaceutical compositions consisting essentially of bictegravir, or a pharmaceutically acceptable salt thereof, and the compound of formula III, or a pharmaceutically acceptable salt thereof, and a pharmaceutically acceptable carrier, diluent or excipient are provided.

In one embodiment, pharmaceutical compositions consisting of bictegravir, or a pharmaceutically acceptable salt thereof, and the compound of formula III, or a pharmaceutically acceptable salt thereof, and one or more pharmaceutically acceptable carriers, diluents or excipients are provided.

In yet another embodiment, combinations comprising bictegravir, or a pharmaceutically acceptable salt thereof, and a compound of formula III, or a pharmaceutically acceptable salt thereof, are provided.

In yet another embodiment, combinations consisting essentially of bictegravir, or a pharmaceutically acceptable salt thereof; and a compound of formula III, or a pharmaceutically acceptable salt thereof, are provided.

In yet another embodiment, combinations consisting of bictegravir, or a pharmaceutically acceptable salt thereof; and a compound of formula III, or a pharmaceutically acceptable salt thereof; and one or more pharmaceutically acceptable carriers, diluents or excipients are provided.

Another embodiment provides further administration of at least one antiretroviral compound.

In certain embodiments, a method for treating an HIV infection in a human, comprising administering to the human a therapeutically effective amount of bictegravir, or a pharmaceutically acceptable salt thereof, and the compound of formula II, or a pharmaceutically acceptable salt thereof, in combination with a therapeutically effective amount of one or more (e.g., one, two, three, one or two, or one to three) additional therapeutic agents.

One embodiment of the invention provides a method for preventing HIV infection in a human comprising administering to the human a therapeutically effective amount of bictegravir, or a pharmaceutically acceptable salt thereof; and a therapeutically effective amount of the compound of formula II, or a pharmaceutically acceptable salt thereof, in combination with atherapeutically effective amount of one or more additional therapeutic agents that are suitable for treating an HIV infection.

In another embodiment, the present disclosure provides a method for preventing an HIV infection, comprising administering to a human a therapeutically effective amount of bictegravir, or a pharmaceutically acceptable salt thereof, and the compound of formula II, or a pharmaceutically acceptable salt thereof, in combination with atherapeutically effective amount of one or more additional therapeutic agents that are suitable for treating an HIV infection.

In certain embodiments, a method for treating an HIV infection in a human, comprising administering to the human a therapeutically effective amount of bictegravir, or a pharmaceutically acceptable salt thereof, and the compound of formula III, or a pharmaceutically acceptable salt thereof, in combination with a therapeutically effective amount of one or more (e.g., one, two, three, one or two, or one to three) additional therapeutic agents.

One embodiment of the invention provides a method for preventing HIV infection in a human comprising administering to the human a therapeutically effective amount of bictegravir, or a pharmaceutically acceptable salt thereof; and a therapeutically effective amount of the compound of formula III, or a pharmaceutically acceptable salt thereof, in combination with atherapeutically effective amount of one or more additional therapeutic agents that are suitable for treating an HIV infection.

In one embodiment, pharmaceutical compositions comprising bictegravir, or a pharmaceutically acceptable salt thereof, and the compound of formula II, or a pharmaceutically acceptable salt thereof, in combination with one or more (e.g., one, two, three, one to two, or one to three) additional therapeutic agents, and a therapeutically acceptable carrier, diluent or excipient are provided.

In a further embodiment, kits comprising bictegravir, or a pharmaceutically acceptable salt thereof, and the compound of formula II, or a pharmaceutically acceptable salt thereof, in oral and/or injectible, or parenteral dosage forms are provided. Certain such kits comprise bictegravir in a syringe dosage, tablet dosage form, or an injectible form and the compound of formula II in a syringe dosage or tablet dosage form.

According to another embodiment, there is provided the use of a combination, including bictegravir, or a pharmaceutically acceptable salt thereof, and the compound of formula II, or a pharmaceutically acceptable salt thereof, for the treatment of HIV.

In one embodiment, pharmaceutical compositions comprising bictegravir, or a pharmaceutically acceptable salt thereof, and the compound of formula III, or a pharmaceutically acceptable salt thereof, in combination with one or more (e.g., one, two, three, one to two, or one to three) additional therapeutic agents, and a therapeutically acceptable carrier, diluent or excipient are provided.

In a further embodiment, kits comprising bictegravir, or a pharmaceutically acceptable salt thereof, and the compound of formula III, or a pharmaceutically acceptable salt thereof, in oral and/or injectible, or parenteral dosage forms are provided. Certain such kits comprise bictegravir in a syringe dosage, tablet dosage form, or injectible dosage form, and the compound of formula III in a syringe dosage or tablet dosage form.

According to another embodiment, there is provided the use of a combination, including bictegravir, or a pharmaceutically acceptable salt thereof, and the compound of formula III, or a pharmaceutically acceptable salt thereof, for the treatment of HIV.

In the above embodiments, the additional therapeutic agent may be an anti-HIV agent. For example, in some embodiments, the additional therapeutic agent is chosen from: HIV protease inhibitors, HIV non-nucleoside or non-nucleotide inhibitors of reverse transcriptase, MK-8591 (EFdA) HIV nucleoside or nucleotide inhibitors of reverse transcriptase, HIV integrase inhibitors, HIV non-catalytic site (or allosteric) integrase inhibitors, HIV entry inhibitors (e.g., combinectin, CCR5 inhibitors, gp41 inhibitors (i.e., fusion inhibitors) and CD4 attachment inhibitors), CXCR4 inhibitors, gp120 inhibitors, G6PD and NADH-oxidase inhibitors, HIV vaccines, latency reversing agents (e.g., histone deacetylase inhibitors, proteasome inhibitors, protein kinase C (PKC) activators, and BRD4 inhibitors), compounds that target the HIV capsid (“capsid inhibitors”; e.g., capsid polymerization inhibitors or capsid disrupting compounds, HIV nucleocapsid p7 (NCp7) inhibitors, HIV p24 capsid protein inhibitors), pharmacokinetic enhancers, immune-based therapies, e.g., immune checkpoint inhibitors or agonists, Pd-1 modulators, Pd-L1 modulators, CTLA4 modulators, ICOS modulators, OX40 modulators, and the like, toll-like receptors modulators, IL-15 agonists, HIV antibodies, bispecific antibodies and “antibody-like” therapeutic proteins (e.g., DARTs®, DUOBODIES®, BITES®, XmAbs®, TandAbs®, Fab derivatives) including those targeting HIV gp120 or gp41, combination drugs for HIV, HIV p 17 matrix protein inhibitors, IL-13 antagonists, peptidylprolyl cis-trans isomerase A modulators, protein disulfide isomerase inhibitors, complement C5a receptor antagonists, DNA methyltransferase inhibitor, HIV vif gene modulators, Vif dimerization antagonists, HIV-1 viral infectivity factor inhibitors, TAT protein inhibitors, HIV-1 Nef modulators, Hck tyrosine kinase modulators, mixed lineage kinase-3 (MLK-3) inhibitors, HIV-1 splicing inhibitors, Rev protein inhibitors, integrin antagonists, nucleoprotein inhibitors, splicing factor modulators, COMM domain containing protein 1 modulators, HIV ribonuclease H inhibitors, retrorocyclin modulators, CDK-9 inhibitors, dendritic ICAM-3 grabbing nonintegrin 1 inhibitors, HIV GAG protein inhibitors, HIV POL protein inhibitors, complement Factor H modulators, ubiquitin ligase inhibitors, deoxycytidine kinase inhibitors, cyclin dependent kinase inhibitors proprotein convertase PC9 stimulators, ATP-dependent RNA helicase DDX3X inhibitors, reverse transcriptase priming complex inhibitors, HIV gene therapy, PI3K inhibitors, compounds, such as those disclosed in WO 2013/006738 (Gilead Sciences®), US 2013/0165489 (University of Pennsylvania®), WO 2013/091096A1 (Boehringer Ingelheim®), WO 2009/062285 (Boehringer Ingelheim®), US20140221380 (Japan Tobacco®), US 20140221378 (Japan Tobacco®), WO 2010/130034 (Boehringer Ingelheim®), WO 2013/159064 (Gilead Sciences®), WO 2012/145728 (Gilead Sciences®), W02012/003497 (Gilead Sciences®), W02014/100323 (Gilead Sciences®), W02012/145728 (Gilead Sciences®), W0 2013/159064 (Gilead Sciences®) and WO 2012/003498 (Gilead Sciences®) and WO 2013/006792 (Pharma Resources®), and other drugs for treating HIV, and combinations thereof.

In certain embodiments, the additional therapeutic is chosen from: HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, pharmacokinetic enhancers, and combinations thereof.

In certain embodiments, bictegravir, or a pharmaceutically acceptable salt thereof, and the compound of formula II, or a pharmaceutically acceptable salt thereof, are formulated as a tablet that may optionally contain one or more other compounds useful for treating HIV. In certain embodiments, the tablet can contain another active ingredient for treating HIV, such as HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, pharmacokinetic enhancers, and combinations thereof. In certain embodiments, such tablets are suitable for once daily dosing.

In certain embodiments, bictegravir, or a pharmaceutically acceptable salt thereof, and the compound of formula III, or a pharmaceutically acceptable salt thereof, are formulated as a tablet that may optionally contain one or more other compounds useful for treating HIV. In certain embodiments, the tablet can contain another active ingredient for treating HIV, such as HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, pharmacokinetic enhancers, and combinations thereof. In certain embodiments, such tablets are suitable for once daily dosing.

In other embodiments, the additional therapeutic agent may be chosen from one or more of:

(1) Combination drugs chosen from: ATRIPLA® (efavirenz+tenofovir disoproxil fumarate+emtricitabine), COMPLERA® (EVIPLERA®, rilpivirine+tenofovir disoproxil fumarate+emtricitabine), STRIBILD® (elvitegravir+cobicistat+tenofovir disoproxil fumarate+emtricitabine), lamivudine+nevirapine+zidovu dine, atazanavir sulfate+cobicistat, darunavir+cobicistat, efavirenz+lamivudine+tenofovir disoproxil fumarate, Vacc-4x+romidepsin, APH-0812, raltegravir+lamivudine, KALETRA® (ALUVIA®, lopinavir+ritonavir), atazanavir sulfate+ritonavir, COMBIVIR® (zidovudine+lamivudine, AZT+3TC), EPZICOM® (KIVEXA®, abacavir sulfate+lamivudine, ABC+3TC), TRIZIVIR® (abacavir sulfate+zidovudine+lamivudine, ABC+AZT+3TC), TRUVADA® (tenofovir disoproxil fumarate+emtricitabine, TDF+FTC), tenofovir+lamivudine, atazanavir+cobicistat, doravirine+lamivudine+tenofovir disoproxil fumarate, doravirine+lamivudine+tenofovir disoproxil and lamivudine+tenofovir disoproxil fumarate;

(2) HIV protease inhibitors chosen from: amprenavir, atazanavir, fosamprenavir, fosam-prenavir calcium, indinavir, indinavir sulfate, lopinavir, ritonavir, nelfinavir, nelfinavir mesylate, saquinavir, saquinavir mesylate, tipranavir, brecanavir, darunavir, DG-17, TMB-657 (PPL-100), TMC-310911, and TMB-657;

(3) HIV non-nucleoside or non-nucleotide inhibitors of reverse transcriptase chosen from: delavirdine, delavirdine mesylate, nevirapine, (+), etravirine, dapivirine, doravirine, efavirenz, KM023, VM-1500, lentinan, AIC-292, EFdA (4′-Ethynyl-2-Fluoro-2′-Deoxyadenosine, or otherwise known as MK-8591), ENDURANT® (rilpivirine), lentinan, AIC-292, and KM-023;

(4) HIV nucleoside or nucleotide inhibitors of reverse transcriptase chosen from: VIDEX® and VIDEX® EC (didanosine, ddl), zidovudine, emtricitabine, didanosine, stavudine, zalcitabine, lamivudine, censavudine, abacavir, abacavir sulfate, amdoxovir, elvucitabine, alovudine, phosphazid, fozivudine tidoxil, apricitabine, amdoxovir, KP-1461, fosalvudine tidoxil, tenofovir disoproxil, tenofovir disoproxil fumarate, tenofovir disoproxil hemifumarate, adefovir, adefovir dipivoxil, festinavir, and MK-8591 (EFdA).

(5) HIV integrase inhibitors chosen from: raltegravir, elitegravir, cabotegravir, and dolutegratir (TIVICAVy®);

(6) HIV non-catalytic site, or allosteric, integrase inhibitors (NCINI) chosen from: CX-05168, CX-05045 and CX-14442;

(7) HIV gp41 inhibitors chosen from: enfuvirtide, sifuvirtide and albuvirtide;

(8) HIV entry inhibitors, such as combinectin;

(9) HIV gp120 inhibitors chosen from: Radha-108 (Receptol) and BMS-663068;

(10) CCR5 inhibitors chosen from: aplaviroc, vicriviroc, maraviroc, cenicriviroc, PR0-140, Adaptavir (RAP-101), nifeviroc (TD-0232), TD-0680, TBR-220 (TAK-220) and vMIP (Haimipu);

(11) CD4 attachment inhibitors, such as ibalizumab;

(12) CXCR4 inhibitors chosen from: plerixafor, ALT-1188, vMIP and Haimipu;

(13) Pharmacokinetic enhancers or boosting agents chosen from: cobicistat (TYBOST®), ritonavir (NORVIR®), and SPI-452;

(14) Immune-based therapies chosen from: dermaVir, interleukin-7, lexgenleucel-T (VRX-496), plaquenil (hydroxychloroquine), proleukin (aldesleukin, IL-2), interferon alfa, interferonalfa-2b, interferon alfa-n3, pegylated interferon alfa, interferon gamma, hydroxyurea, mycophenolate mofetil (MPA) and its ester derivative mycophenolate mofetil (MMF), WF-10, ribavirin, IL-2, IL-2 XL, IL-12, polymer polyethyleneimine (PEI), Gepon, VGV-1, MOR-22, toll-like receptors modulators (tlr1, tlr2, tlr3, tlr4, tlr5, tlr6, tlr7, tlr8, tlr9, tlr10, tlr11, tlr12 and tlr13), BMS-936559, rintatolimod and IR-103;

(15) HIV vaccines chosen from: peptide vaccines, recombinant subunit protein vaccines, live vector vaccines, DNA vaccines, virus-like particle vaccines (pseudovirion vaccine), CD4-derived peptide vaccines, vaccine combinations, rgp120 (AIDSVAX®), ALVAC HIV (vCP1521)/AIDSVAX B/E (gp120) (RV144), Remune®, ITV-1, Cantre Vir,Ad5-ENVA-48, DCVax-001 (CDX-2401), PEP-6409, Vacc-4x, Vacc-C5, VAC-3S, multiclade DNA recombinant adenovirus-5 (rAdS), Pennvax-G, YRC-HIV MAB060-00-AB, AVX-101, Tat Oyi vaccine, AVX-201, HIV-LAMP-vax, Ad35, Ad35-GRIN, NAcGM3NSSP ISA-51, poly-ICLC adjuvanted vaccines, Tatlmmune, GTU-multi-HIV (FIT-06), AGS-004, gp140[delta]V2.TVI+MF-59, rVSVIN HIV-1 gag vaccine, SeV-Gag vaccine, AT-20, DNK-4, Ad35-GRIN/ENV, TBC-M4, HIVAX, HIVAX-2, NYVAC-HIV-PT1, NYVAC-HIV-PT4, DNA-HIV-PT123, VIChREPOL®, rAAV1-PG9DP, GOVX-B11, GOVX-B21, ThV-01, TUTI-16, VGX-3300, TVI-HIV-1, Ad-4 (Ad4-env Clade C+Ad4-mGag), EN41-FPA2, PreVaxTat, TL-01, SAV-001, AE-H, MYM-VlO1, CombiHIVvac, ADVAX, MYM-V201, monomeric gp120 HIV-1 subtype C vaccine (Novartis®), MVA-CMDR, MVATG-17401, ETV-01, CDX-1401, rcAd26.MOS1.HIV-Env, and DNA-Ad5 gag/pol/nef/nev (HVTN505);

(16) HIV antibodies, bispecific antibodies and “antibody-like” therapeutic proteins (such as DARTs®, Duo-bodies®, Bites®, XmAbs®, TandAbs®, Fab derivatives), including BMS-936559, TMB-360 and those targeting HIV gp120 or gp41 are chosen from: bavituximab, UB-421, C2F5, C2G12, C4E10, C2F5+C2G12+C4E10, 3-BNC-117, KD-247, PGT145, PGT121, MDXOlO (ipilimumab), A32, 7B2, VRC01 (disclosed in WO 2011/038290), VRC01-LS (disclosed in WO 2012/106578), VRC-07 (disclosed in WO 2013/086533), and VRC07-523 (disclosed in WO 2015/048770);

(17) latency-reversing agents chosen from: histone deacetylase inhibitors such as Romidepsin, vorinostat, panobinostat; proteasome inhibitors, such as VELCADE®; protein kinase C (PKC) activators such as Indolactam, prostratin, ingenol B and DAG-lactones, lonomycin, GSK-343, PMA, SAHA, BRD4 inhibitors, IL-15, JQ1, amphotericin B, and disulfram;

(18) HIV nucleocapsid p7 (NCp7) inhibitors, such as azodicarbonamide;

(19) P13K inhibitors chosen from: idelalisib, AZD-8186, buparlisib, CLR-457, pictilisib, neratinib, rigosertib, rigosertib sodium, EN-3342, TGR-1202, alpelisib, duvelisib, UCB-5857, taselisib, XL-765, gedatolisib, VS-5584, copanlisib, CAI orotate, perifosine, RG-7666, GSK-2636771, DS-7423, panulisib, GSK-2269557, GSK-2126458, CUDC-907, PQR-309, INCB-040093, pilaralisib, BAY-1082439, puquitinib mesylate, SAR-245409, AMG-319, RP-6530, ZSTK-474, MLN-1117, SF-1126, RV-1729, sonolisib, LY-3023414, SAR-260301 and CLR-1401;

(20) the compounds disclosed in WO 2004/096286 (Gilead Sciences), WO 2006/110157 (Gilead Sciences®), WO 2006/015261 (Gilead Sciences®), WO 2013/006738 (Gilead Sciences®), US 2013/0165489 (University of Pennsylvania®), US20140221380 (Japan Tobacco®), US20140221378 (Japan Tobacco®), WO 2013/006792 (Pharma Resources®), WO 2009/062285 (Boehringer Ingelheim®), WO 2010/130034 (Boehringer Ingelheim®), WO 2013/091096A1 (Boehringer Ingelheim®), WO 2013/159064 (Gilead Sciences), WO 2012/145728 (Gilead Sciences®), W02012/003497 (Gilead Sciences®), W02014/100323 (Gilead Sciences®), W02012/145728 (Gilead Sciences®), W02013/159064 (Gilead Sciences®) and WO 2012/003498 (Gilead Sciences®); and

(21) other drugs for treating HIV chosen from: REP 9, cytolin, CYT-107, alisporivir, BanLec, MK-8507, AG-1105, TR-452, MK-8591, REP 9, NOV-205, IND-02, metenkefalin, PGN-007, Acemannan, Gamimune, SCY-635, prolastin, 1,5-dicaffeoylquinic acid, BIT-225, RPI-MN, VSSP, Hlviral, IM0-3100, SB-728-T, RPI-MN, VIR-576, HGTV-43, MK-1376, rHIV7-sh1-TAR-CCR5RZ, MazF gene therapy, BlockAide, ABX-464, SCY-635, naltrexone, AAV-eCD4-Ig gene therapy, TEV-90110, TEV-90112, deferiprone, and PA-1050040 (PA-040).

In certain embodiments, bictegravir, or a pharmaceutically acceptable salt thereof, and the compound of formula II, or a pharmaceutically acceptable salt thereof, are combined with one, two, three, four or more additional therapeutic agents. The one, two, three, four or more additional therapeutic agents can be different therapeutic agents selected from the same class of therapeutic agents, and/or they can be selected from different classes of therapeutic agents.

In certain embodiments, bictegravir, or a pharmaceutically acceptable salt thereof, and the compound of formula III, or a pharmaceutically acceptable salt thereof, are combined with one, two, three, four or more additional therapeutic agents. The one, two, three, four or more additional therapeutic agents can be different therapeutic agents selected from the same class of therapeutic agents, and/or they can be selected from different classes of therapeutic agents.

In a particular embodiment, bictegravir, or a pharmaceutically acceptable salt thereof, are combined with one, two, three, four or more additional therapeutic agents selected from raltegravir, TRUVADA® (tenofovir disoproxil fumarate+emtricitabine, TDF+FTC), maraviroc, enfuvirtide, EPZICOM® (KIVEXA®, abacavir sulfate+lamivu-dine, ABC+3TC), TRIZIVIR® (abacavir sulfate+zidovudine+lamivudine, ABC+AZT+3TC), adefovir, adefovir dipivoxil, STRIBILD® (elvitegravir+cobicistat+tenofovir disoproxil fumarate+emtricitabine), raltegravir+lamivudine, COMPLERA® (EVIPLERA®, rilpivirine+tenofovir disoproxil fumarate+emtricitabine), Cobicistat, ATRIPLA® (efavirenz+tenofovir disoproxil fumarate+emtricitabine), atazanavir sulfate+cobicistat, atazanavir+cobicistat, darunavir+cobicistat, atazanavir, atazanavir sulfate, elvitegravir, ALUVIA®, (KALETRA®, lopinavir+ritonavir), ritonavir, emtricitabine, atazanavir sulfate+ritonavir, darunavir, lamivudine, Prolastin, fosamprenavir, fosamprenavir calcium, efavirenz, COMBIVIR® (zidovudine+lamivudine, AZT+3TC), etravirine, nelfinavir, nelfininavir mesylate, interferon, didanosine, stavudine, indinavir, indinavir sulfate, tenofovir+lamivudine, zidovudine, nevirapine, saquinavir, saquinavir mesylate, aldesleukin, zalcitabine, tipranavir, amprenavir, delavirdine, delavirdine mesylate, Radha-108 (RECEPTOL®), Rivira, lamivudine+tenofovir disoproxil fumarate, efavirenz+lamivudine+tenofovir disoproxil fumarate, phosphazid, lamivudine+nevirapine+zidovudine, abacavir, abacavir sulfate, tenofovir, tenofovir, tenofovir disoproxil and tenofovir disoproxil fumarate.

In one embodiment, bictegravir, or a pharmaceutically acceptable salt thereof, and the compound of formula II, or a pharmaceutically acceptable salt thereof, are administered with either an agent chosen from emtricitabine and lamivudine. In certain embodiments, when bictegravir, or a pharmaceutically acceptable salt thereof, and the compound of formula II, or a pharmaceutically acceptable salt thereof, are combined with one or more additional therapeutic agents as described above, the components of the composition are administered as a simultaneous or sequential regimen. When administered sequentially, either combination may be administered in two or more administrations.

In certain embodiments, bictegravir, or a pharmaceutically acceptable salt thereof, and the compound of formula II, or a pharmaceutically acceptable salt thereof, thereof, are combined with one or more additional therapeutic agents, in a unitary dosage form for simultaneous administration to a human, for example, as a solid dosage form for oral administration. In certain embodiments, bictegravir, or a pharmaceutically acceptable salt thereof, and the compound of formula II, or a pharmaceutically acceptable salt thereof, are administered with one or more additional therapeutic agents. Co-administration of bictegravir, or a pharmaceutically acceptable salt thereof, and the compound of formula II, or a pharmaceutically acceptable salt thereof, with one or more additional therapeutic agents generally refers to simultaneous or sequential administration of a compound disclosed herein and one or more additional therapeutic agents, such that therapeutically effective amounts of the compound disclosed herein and one or more additional therapeutic agents are both present in the body of the human.

Co-administration includes administration of unit dosages of bictegravir, or a pharmaceutically acceptable salt thereof, and the compound of formula II, or a pharmaceutically acceptable salt thereof, before or after administration of unit dosages of one or more additional therapeutic agents, for example, administration of bictegravir, or a pharmaceutically acceptable salt thereof, and the compound of formula II, or a pharmaceutically acceptable salt thereof, within seconds, minutes, or hours of the administration of one or more additional therapeutic agents. For example, in some embodiments, a unit dose of bictegravir, or a pharmaceutically acceptable salt thereof, and the compound of formula II, or a pharmaceutically acceptable salt thereof, is administered first, followed within seconds or minutes by administration of a unit dose of one or more additional therapeutic agents. Alternatively, in other embodiments, a unit dose of one or more additional therapeutic agents is administered first, followed by administration of a unit dose of bictegravir, or a pharmaceutically acceptable salt thereof, and the compound of formula II, or a pharmaceutically acceptable salt thereof, within seconds or minutes. In some embodiments, a unit dose of bictegravir, or a pharmaceutically acceptable salt thereof; and the compound of formula II, or a pharmaceutically acceptable salt thereof, is administered first, followed, after a period of hours (e.g., 1-12 hours), by administration of a unit dose of one or more additional therapeutic agents. In other embodiments, a unit dose of one or more additional therapeutic agents is administered first, followed, after a period of hours (e.g., 1-12 hours), by administration of a unit dose of bictegravir, or a pharmaceutically acceptable salt thereof, and the compound of formula II, or a pharmaceutically acceptable salt thereof.

In certain embodiments, bictegravir, or a pharmaceutically acceptable salt thereof, and the compound of formula II, or a pharmaceutically acceptable salt thereof, are administered orally.

In another embodiment, the combination of bictegravir, or a pharmaceutically acceptable salt thereof, and the compound of formula II, or a pharmaceutically acceptable salt thereof, is administered to the human once a day.

In another embodiment, the combination of bictegravir, or a pharmaceutically acceptable salt thereof, and the compound of formula II, or a pharmaceutically acceptable salt thereof, is administered to the human twice a day.

In another embodiment, bictegravir, or a pharmaceutically acceptable salt thereof, is administered to the human at about 5 mg, about 10 mg, about 25 mg, about 30 mg, about 35 mg, about 40 mg, about 45 mg dose, about 50 mg, about 55 mg, about 60 mg, about 65 mg, about 70 mg, about 75 mg, about 80 mg, about 85 mg, about 90 mg, about 95 mg or about 100 mg, once, twice, or three times a day.

In another embodiment, bictegravir, or a pharmaceutically acceptable salt thereof, is administered to the human at about 5 mg, about 10 mg, about 25 mg to 100 mg, at about 25 mg to 75 mg, at about 35 mg to 65 mg or about 45 mg to 55 mg, once or twice per day. In another embodiment, bictegravir, or a pharmaceutically acceptable salt thereof, is administered to the human at about 50 mg, once or twice per day.

In a further embodiment, the compound of formula II, or a pharmaceutically acceptable salt thereof, is administered to the human at about 1 mg, about 5 mg, about 10 mg, about 15 mg, about 20 mg, about 25 mg dose, about 30 mg, about 35 mg, about 40 mg, about 45 mg, about 50 mg, about 60 mg, about 100 mg, about 120 mg, about 200 mg, about 250 mg, about 300 mg, about 350 mg, about 400 mg, about 450 mg, or about 500 mg once, twice or three times a day. In another embodiment, the compound of formula II, or a pharmaceutically acceptable salt thereof, is administered to the human at about 1 mg to 50 mg or at about 5 mg to 25 mg once per day. In another embodiment, the compound of formula II, or a pharmaceutically acceptable salt thereof, is administered to the human at about 5 mg, once per day. In another embodiment, the compound of formula II, or a pharmaceutically acceptable salt thereof, is administered to the human at about 10 mg, once per day. In another embodiment, the compound of formula II, or a pharmaceutically acceptable salt thereof, is administered to the human at about 25 mg, once per day. In a further embodiment, the compound of formula II, or a pharmaceutically acceptable salt thereof is administered to the human at about 5 mg, about 10 mg, about 25 mg, about 50 mg, about 75 mg, about 100 mg, about 125 mg, about 150 mg, about 175 mg, or about 200 mg, optionally in combination with the boosting agent, NORVIR®.

In another embodiment, this invention relates to a combination comprising bictegravir, or a pharmaceutically acceptable salt thereof; and the compound of formula II, or a pharmaceutically acceptable salt thereof.

In another embodiment, this invention relates to the above combination for use in medical therapy.

In another embodiment, this invention relates to the above combination for use in the treatment of HIV.

In yet another embodiment, this invention relates to pharmaceutical composition comprising a combination comprising bictegravir, or a pharmaceutically acceptable salt thereof and the compound of formula II, or a pharmaceutically acceptable salt thereof.

In another embodiment, this invention relates to pharmaceutical composition comprising a combination comprising bictegravir, or a pharmaceutically acceptable salt thereof; and the compound of formula II, or a pharmaceutically acceptable salt thereof in association with one or more pharmaceutically acceptable carriers therefor.

In a further embodiment, the present invention provides for the use of bictegravir, or a pharmaceutically acceptable salt thereof; and the compound of formula II, or a pharmaceutically acceptable salt thereof, for the manufacture of a medicament for the treatment of HIV.

In one embodiment, bictegravir, or a pharmaceutically acceptable salt thereof, and the compound of formula III, or a pharmaceutically acceptable salt thereof, are administered with an agent chosen from: emtricitabine and lamivudine. In certain embodiments, when bictegravir, or a pharmaceutically acceptable salt thereof, and the compound of formula II, or a pharmaceutically acceptable salt thereof, are combined with one or more additional therapeutic agents as described above, the components of the composition are administered as a simultaneous or sequential regimen. When administered sequentially, either combination may be administered in two or more administrations.

In certain embodiments, bictegravir, or a pharmaceutically acceptable salt thereof, and the compound of formula III, or a pharmaceutically acceptable salt thereof, thereof, are combined with one or more additional therapeutic agents, in a unitary dosage form for simultaneous administration to a human, for example, as a solid dosage form for oral administration. In certain embodiments, bictegravir, or a pharmaceutically acceptable salt thereof, and the compound of formula II, or a pharmaceutically acceptable salt thereof, are administered with one or more additional therapeutic agents. Co-administration of bictegravir, or a pharmaceutically acceptable salt thereof, and the compound of formula III, or a pharmaceutically acceptable salt thereof, with one or more additional therapeutic agents generally refers to simultaneous or sequential administration of a compound disclosed herein and one or more additional therapeutic agents, such that therapeutically effective amounts of the compound disclosed herein and one or more additional therapeutic agents are both present in the body of the human.

Co-administration includes administration of unit dosages of bictegravir, or a pharmaceutically acceptable salt thereof, and the compound of formula III, or a pharmaceutically acceptable salt thereof, before or after administration of unit dosages of one or more additional therapeutic agents, for example, administration of bictegravir, or a pharmaceutically acceptable salt thereof, and the compound of formula III, or a pharmaceutically acceptable salt thereof, within seconds, minutes, or hours of the administration of one or more additional therapeutic agents. For example, in some embodiments, a unit dose of bictegravir, or a pharmaceutically acceptable salt thereof, and the compound of formula III, or a pharmaceutically acceptable salt thereof, is administered first, followed within seconds or minutes by administration of a unit dose of one or more additional therapeutic agents. Alternatively, in other embodiments, a unit dose of one or more additional therapeutic agents is administered first, followed by administration of a unit dose of bictegravir, or a pharmaceutically acceptable salt thereof, and the compound of formula III, or a pharmaceutically acceptable salt thereof, within seconds or minutes. In some embodiments, a unit dose of bictegravir, or a pharmaceutically acceptable salt thereof; and the compound of formula III, or a pharmaceutically acceptable salt thereof, is administered first, followed, after a period of hours (e.g., 1-12 hours), by administration of a unit dose of one or more additional therapeutic agents. In other embodiments, a unit dose of one or more additional therapeutic agents is administered first, followed, after a period of hours (e.g., 1-12 hours), by administration of a unit dose of bictegravir, or a pharmaceutically acceptable salt thereof, and the compound of formula III, or a pharmaceutically acceptable salt thereof.

In certain embodiments, bictegravir, or a pharmaceutically acceptable salt thereof, and the compound of formula III, or a pharmaceutically acceptable salt thereof, are administered orally.

In another embodiment, the combination of bictegravir, or a pharmaceutically acceptable salt thereof, and the compound of formula III, or a pharmaceutically acceptable salt thereof, is administered to the human once a day.

In another embodiment, the combination of bictegravir, or a pharmaceutically acceptable salt thereof, and the compound of formula III, or a pharmaceutically acceptable salt thereof, is administered to the human twice a day.

In a further embodiment, the compound of formula III, or a pharmaceutically acceptable salt thereof, is administered to the human at about 1 mg, about 5 mg, about 10 mg, about 15 mg, about 20 mg, about 25 mg dose, about 30 mg, about 35 mg, about 40 mg, about 45 mg, about 50 mg, about 60 mg, about 100 mg, about 120 mg, about 200 mg, about 250 mg, about 300 mg, about 350 mg, about 400 mg, about 450 mg, or about 500 mg once, twice or three times a day. In another embodiment, the compound of formula III, or a pharmaceutically acceptable salt thereof, is administered to the human at about 1 mg to 50 mg or at about 5 mg to 25 mg once per day. In another embodiment, the compound of formula III, or a pharmaceutically acceptable salt thereof, is administered to the human at about 5 mg, once per day. In another embodiment, the compound of formula III, or a pharmaceutically acceptable salt thereof, is administered to the human at about 10 mg, once per day. In another embodiment, the compound of formula III, or a pharmaceutically acceptable salt thereof, is administered to the human at about 25 mg, once per day. In a further embodiment, the compound of formula III, or a pharmaceutically acceptable salt thereof is administered to the human at about 5 mg, about 10 mg, about 25 mg, about 50 mg, about 75 mg, about 100 mg, about 125 mg, about 150 mg, about 175 mg, or about 200 mg, optionally, in combination with the boosting agent, NORVIR®.

In another embodiment, this invention relates to a combination comprising bictegravir, or a pharmaceutically acceptable salt thereof; and the compound of formula III, or a pharmaceutically acceptable salt thereof.

In another embodiment, this invention relates to the above combination for use in medical therapy.

In another embodiment, this invention relates to the above combination for use in the treatment of HIV.

In yet another embodiment, this invention relates to pharmaceutical composition comprising a combination comprising bictegravir, or a pharmaceutically acceptable salt thereof and the compound of formula III, or a pharmaceutically acceptable salt thereof.

In another embodiment, this invention relates to pharmaceutical composition comprising a combination comprising bictegravir, or a pharmaceutically acceptable salt thereof; and the compound of formula III, or a pharmaceutically acceptable salt thereof in association with one or more pharmaceutically acceptable carriers therefor.

In a further embodiment, the present invention provides for the use of bictegravir, or a pharmaceutically acceptable salt thereof; and the compound of formula III, or a pharmaceutically acceptable salt thereof, for the manufacture of a medicament for the treatment of HIV.

Definitions

As used herein, the term “co-administer” refers to administration of two or more agents within a 24-hour period of each other, for example, as part of a clinical treatment regimen. In other embodiments, “co-administer” refers to administration of two or more agents within 2 hours of each other. In other embodiments, “co-administer” refers to administration of two or more agents within 30 minutes of each other. In other embodiments, “co-administer” refers to administration of two or more agents within 15 minutes of each other. In other embodiments, “co-administer” refers to administration at the same time, either as part of a single formulation or as multiple formulations that are administered by the same or different routes.

“Bictegravir”, formula I, is:

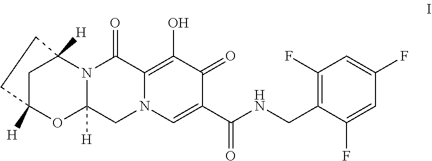 [SMILES: O=C(NCc1c(F)cc(F)cc1F)c1cn2c(c(O)c1=O)C(=O)N1[C@H]3CC[C@H](C3)O[C@@H]1C2]

Methods for preparing bictegravir are described in U.S. Pat. No. 8,129,385. Bictegravir is currently in clinical trials for for the treatment and prevention of HIV.

“Emtricitabine” or “FTC” refers to (2R,5S,cis)-4-amino-5-fluoro-1-(2-hydroxymethyl-1,3-oxathiolan-5-yl)-(1H)-pyrimidin-2-one.

“Pharmaceutically acceptable salt” refers to a salt of a compound that is pharmaceutically acceptable and that possesses (or can be converted to a form that possesses) the desired pharmacological activity of the parent compound. Examples of “pharmaceutically acceptable salts” of the compounds disclosed herein include salts derived from an appropriate base, such as an alkali metal (for example, sodium), an alkaline earth metal (for example, magnesium), ammonium and NX₄⁺ (wherein X is C₁-C₄ alkyl). Pharmaceutically acceptable salts of a nitrogen atom or an amino group include, for example, salts of organic carboxylic acids such as acetic, benzoic, camphorsulfonic, citric, glucoheptonic, gluconic, lactic, fumaric, tartaric, maleic, malonic, malic, mandelic, isethionic, lactobionic, succinic, 2-napththalenesulfonic, oleic, palmitic, propionic, stearic, and trimethylacetic acids; organic sulfonic acids, such as methanesulfonic, ethanesulfonic, benzenesulfonic and p-toluenesulfonic acids; and inorganic acids, such as hydrochloric, hydrobromic, sulfuric, nitric, phosphoric and sulfamic acids. Pharmaceutically acceptable salts of a compound of a hydroxy group include the anion of said compound in combination with a suitable cation, such as Na⁺ and NX₄⁺ (wherein X is independently selected from H or a C₁-C₄ alkyl group). Pharmaceutically acceptable salts also include salts formed when an acidic proton present in the parent compound is replaced by either a metal ion, e.g., an alkali metal ion, an alkaline earth ion, or an aluminum ion; or coordinates with an organic base such as diethanolamine, triethanolamine, N-methylglucamine and the like. Also included in this definition are ammonium and substituted or quaternized ammonium salts. Representative non-limiting lists of pharmaceutically acceptable salts can be found in Berge, et al., J. Pharma Sci., 66(1), 1-19 (1977), and REMINGTON: THE SCIENCE AND PRACTICE OF PHARMACY, R. HENDRICKSON, ed., 21st edition, Lippincott, Williams & Wilkins, Philadelphia, Pa., (2005), at p. 732, Table 38-5.

For therapeutic use, salts of active ingredients of the compounds disclosed herein will typically be pharmaceutically acceptable, i.e., they will be salts derived from a physiologically acceptable acid or base. However, salts of acids or bases that are not pharmaceutically acceptable may also find use, for example, in the preparation or purification of bictegravir or another compound of the invention. All salts, whether or not derived from a physiologically acceptable acid or base, are within the scope of the present invention.

Metal salts typically are prepared by reacting the metal hydroxide with a compound of this invention. Examples of metal salts that are prepared in this way are salts containing Li⁺, Na⁺, and K⁺. A less soluble metal salt can be precipitated from the solution of a more soluble salt by addition of the suitable metal compound.

In addition, salts may be formed from acid addition of certain organic and inorganic acids, e.g., HCl, HBr, H₂SO₄, H₃PO₄ or organic sulfonic acids, to basic centers, typically amines. Finally, it is to be understood that the compositions herein comprise compounds disclosed herein in their un-ionized, as well as zwitterionic form, and combinations with stoichiometric amounts of water as in hydrates.

In another example, a zwitterionic compound is encompassed herein by referring to a compound that is known to form zwitterions, even if it is not explicitly named in its zwitterionic form. Terms such as zwitterion, zwitterions, and their synonyms zwitterionic compound(s) are standard IUPAC-endorsed names that are well known and part of standard sets of defined scientific names. In this regard, the name zwitterion is assigned the name identification, CHEBI:27369, by the Chemical Entities of Biological Interest (ChEBI) dictionary of molecular entities. (See, for example, its on-line version at http://www.ebi.ac.uk/chebi/init.do). As generally well known, a zwitterion or zwitterionic compound is a neutral compound that has formal unit charges of opposite sign. Sometimes these compounds are referred to by the term “inner salts”. Other sources refer to these compounds as “dipolar ions”, although the latter term is regarded by still other sources as a misnomer. As a specific example, aminoethanoic acid (the amino acid glycine) has the formula H₂NCH₂COOH, and it exists in some media (in this case in neutral media) in the form of the zwitterions ⁺H₃NCH2COO⁻. Zwitterions, zwitterionic compounds, inner salts and dipolar ions in the known and well established meanings of these terms are within the scope of this invention, as would in any case be so appreciated by those of ordinary skill in the art. Because there is no need to name each and every embodiment that would be recognized by those of ordinary skill in the art, no structures of the zwitterionic compounds that are associated with the compounds of this invention are given explicitly herein. They are, however, part of the embodiments of this invention. No further examples in this regard are provided herein because these interactions and transformations in a given medium are known by any one of ordinary skill in the art.

“Therapeutically effective amount” refers to that amount of the compound being administered that will prevent a condition, or will reduce to some extent one or more of the symptoms of the disorder being treated. Pharmaceutical compositions suitable for use herein include compositions, wherein the active ingredients are contained in an amount sufficient to achieve the intended purpose. Determination of a therapeutically effective amount is well within the capability of those skilled in the art, especially in light of the detailed disclosure provided herein.

As used herein, “prevention”, refers to reducing the risk of infection or exhibiting signs or symptoms of such infection in a human at high risk so has not been pre-treated.

As used herein, “treatment”, refers to inhibition, reduction, elimination or alleviation of a disease.

Combinations and Methods of Treatment

A method for the treatment or prophylaxis of diseases, disorders, and conditions is provided herein. An example of a disease, disorder, or condition includes, but is not limited to, a retrovirus infection, or a disease, disorder, or condition associated with a retrovirus infection. Retroviruses are RNA viruses and are generally classified into the alpharetrovirus, betaretrovirus, deltaretrovirus, epsilonretrovirus, gammaretrovirus, lentivirus, and spumavirus families. Examples of retroviruses include, but are not limited to, human immunodeficiency virus (HIV).

The active agents of the disclosed combination therapy may be administered to a human in any conventional manner. While it is possible for the active agents to be administered as compounds, they are preferably administered as a pharmaceutical composition that can include contact with an acid or base, either in an ionic salt form or in contact with the base or acid (i.e., co-formers) without sharing ions. The salt, acid or base co-former, carrier, or diluent should be acceptable, in the sense of being compatible with the other ingredients and not deleterious to the recipient thereof. Examples of carriers or diluents for oral administration include, but are not limited to: cornstarch, lactose, magnesium stearate, talc, microcrystalline cellulose, stearic acid, povidone, crospovidone, dibasic calcium phosphate, sodium starch glycolate, hydroxypropyl cellulose (e.g., low substituted hydroxypropyl cellulose), hydroxypropylmethyl cellulose (e.g., hydroxypropylmethyl cellulose 2910), sodium lauryl sulfate, mannitol, sodium stearyl fumarate, and talc. Examples of salts and acid or base co-formers include fumarate, hemifumarate, sodium, and hydrochloride.

The pharmaceutical compositions may be prepared by any suitable method, such as those methods well known in the art of pharmacy, for example, methods such as those described in Gennaro, et al., REMINGTON'S PHARMACEUTICAL SCIENCES (18th ed., Mack Publishing Co., 1990), especially “Part 8: Pharmaceutical Preparations and their Manufacture”. Such methods include the step of bringing into association the compounds with the carrier or diluents and, optionally, one or more accessory ingredients. Such accessory ingredients include, but are not limited to: fillers, binders, excipients, disintegrants, lubricants, colorants, flavoring agents, sweeteners, preservatives (e.g., antimicrobial preservatives), suspending agents, thickening agents, emulsifying agents, and/or wetting agents.

In practice, the amount of each compound to be administered ranges from about 0.001 to 100 mg per kg of body weight, such total dose being given at one time or in divided doses. Each compound will be administered as a formulation in association with one or more pharmaceutically acceptable excipients. Alternatively, both compounds will be combined and administered as a formulation in association with one or more pharmaceutically acceptable excipients. The choice of excipient will, to a large extent, depend up on factors such as the particular mode of administration, the effect of the excipient on solubility and stability, and the nature of the dosage form. Such compositions and methods for their preparation may be found, for example, in REMINGTON'S PHARMACEUTICAL SCIENCES (19th Edition, Mack Publishing Company, 1995).

In the following description of the example, specific embodiments in which the invention may be practiced are described. These embodiments are described in sufficient detail to enable those skilled in the art to practice the invention. Other embodiments may be utilized and logical, and other changes may be made without departing from the scope of the invention. The following detailed description is, therefore, not to be taken in a limiting sense, and the scope of the invention is defined only by the appended claims, along with the full scope of equivalents to which such claims are entitled.

### EXAMPLES

### Example 1: HIV Cell Line Assay

The HIV cell line experiments taught by Kobayashi, et al., Antimicrobial Agents and Chemotherapy, 55: 814-815 (2011) are followed to test the antiviral abilities of the disclosed and claimed combination of bictegravir and the compound of formula II, or bictegravir and the compound of formula II, as compared with the antiviral abilities of each compound alone.

Bictegravir (the compound of formula I), the compound of formula II, and the compound of formula III are all synthesized by or on behalf of GlaxoSmithKline® and ViiV Healthcare Co®. The structures of each of these compounds are shown above.

### Example 2: Cells and Viruses

Cells of MT-4, a human T-cell leukemia virus type 1 (HTLV-1)-transformed human T-cell line, are maintained as described previously [12]. 293T cells are maintained in Dulbecco's modified Eagle medium (DMEM)-F-12 medium containing 10% fetal bovine serum (FBS). Peripheral blood mononuclear cells (PBMCs) are derived from whole blood samples obtained from HIV-negative donors. PBMCs are separated from whole blood by density gradient centrifugation with Ficoll-Paque Plus® (GE Healthcare®, Waukesha, Wis.) according to the manufacturer's instructions and are stimulated by the addition of either 20 U/ml of interleukin-2 (IL-2) or 10% natural T-cell growth factor (ZeptoMetrix®, Buffalo, N.Y.) plus 5 to 10 μg/ml of phytohemagglutinin (PHA). Molt-4 cells persistently infected with HIV-1 IIIB and MT-2 cells [16] are obtained from S. Harada (Kumamoto University, Kumamoto, Japan). HeLa-CD4 cells containing an HIV-1 long terminal repeat (LTR)-driven β-galactosidase reporter gene have been described previously [20]. MAG1-CCR5 cells have been described previously [9]). HIV-1 strain IIIB is derived from cell-free supernatants of cultures of the chronically infected cell line, H93B (H9/HTLV-IIIB). HIV-1 strain Ba-L is purchased from Advanced Biotechnologies Inc.® (Columbia, Md.) and is expanded in PHA-activated PBMCs, while HIV-1 NL432 [1] is obtained from A. Adachi (Tokushima University, Tokushima, Japan). Plasmid pGJ3-Luci, containing a replication-defective HIV lentiviral vector expressing luciferase [21], is licensed from Christian Jassoy (University of Leipzig), and is used to create stocks of a vesicular stomatitis virus glycoprotein G (VSV-G)-pseudotyped self-inactivating pseudo-HIV (PHIV) lentiviral vector by cotransfection, along with plasmid pVSV-G (Clontech®) into CIP4 cells (a derivative of the 293T human renal epithelial cell line that expresses macrophage scavenger receptor SRA-I to improve adherence to plastic) and harvesting of the cell-free supernatant.

### Example 3: Antiviral Assay in MT-4 Cells

The MT-4 cells generated in Example 2 grow exponentially at a density of 5×10⁵ or 6×10⁵/ml are infected with HIV-1 strain IIIB at a viral multiplicity of infection of 0.001 or a 50% tissue culture infective dose of 4 to 10. The cells are then aliquoted to 96-well plates in the presence of varying concentrations of compounds. After incubation for 4 or 5 days, antiviral activity is determined by a cell viability assay that either measures bioluminescence with a CellTiter-Glo® luminescent reagent (Promega Corporation®, Madison, Wis.) or measured absorbance at 560 and 690 nm using the yellow tetrazolium MTT reagent [3-(4,5-dimethyl-2-thiazolyl)-2,5-diphenyltetrazolium bromide].

### Example 4: Pseudo-HIV Assay

The antiviral activities of bictegravir alone, the compound of formula II alone, and the compound of formula III alone, as well as the combinations of: (1) bictegravir and the compound of formula II; and (2) bictegravir and the compound of formula III are measured in a single-round assay using a self-inactivating PHIV lentiviral vector. CIP4 cells (2×10⁴/well) infected with an amount of PHIV sufficient to produce approximately 50,000 relative light units are added to 96-well black, clear-bottom plates and were incubated for 2 days with all three compounds at varying concentrations. Infected cells are measured as a function of luciferase activity in a luminometer using the Steady-Glo® reagent (Promega Corporation®).

### Example 5: Antiviral Assay in PBMCs

In one 96-well culture plate, PHA- and IL-2-stimulated PBMCs (4×10⁵/well) are pre-incubated with each compound alone, and then for the above combination of compounds, for 1 hour, while HIV-1 strain, Ba-L, is mixed with the same compound in a second plate. An aliquot of the Ba-L-compound mixture is then transferred to the PBMC-compound mixture and is incubated for 7 days. After this incubation, supernatants are assayed for reverse transcriptase (RT) activity by incorporation of [methyl-³H]dTTP to measure viral replication, as previously described [15].

### Example 6: Effects of Human Serum and Serum Proteins

The effect of the presence of human serum albumin (HSA; 20 or 40 mg/ml), α₁-acid glycoprotein (AAG; 2 mg/ml), or human serum (HS; up to 30% or 50% is used, and results were extrapolated up to 100%) on the antiviral activity of each of bictegravir, the compound of formula II, and the compound of formula III is evaluated in the PHIV and MT-4 assay systems. To estimate the effects of the fold shift in protein binding, antiviral activity is tested with the addition of various concentrations of human serum to the HIV-1 IIIB replication assay mixture in MT-4 cells, as previously described [15]. The protein-adjusted half-maximal effective concentration (PA-EC₅₀) is estimated by multiplying the EC₅₀ in PBMCs by the fold shift value. The same experiment is conducted using the combinations of: (1) bictegravir and the compound of formula II; and (2) bictegravir and the compound of formula III.

### Example 7: Combination Antiviral Activity Assay in MT-4 Cells

The in vitro combination activity relationships of: (1) bictegravir alone; (2) the compound of formula II alone; (3) the compound of formula III alone, and the combinations of: (1) bictegravir and the compound of formula II; and (2) bictegravir and the compound of formula III are determined as previously described [39]. Multiple concentrations of the compounds are tested in checkerboard dilution fashion in the presence and absence of dilutions of approved anti-HIV drugs, adefovir, or ribavirin. The assay used HIV-1 IIIB-infected MT-4 cells, and the interaction of the compound combination is analyzed by dose wise additivity-based calculations to quantify deviation from dose wise additivity at the 50% level. Wells containing the top concentration of compounds by themselves are compared to wells with the top concentration of each of the two compound combinations in order to show that combination effects are due to the drugs used, and not simply to toxicity. Assays with the MT-4 system format are run as described previously [15]. Fractional inhibitory concentration (FIC) values in the range of 0.1 to 0.2 indicate weak synergy; values that approach 0.5 indicate strong synergy; and positive values of 0.1 to 0.2 indicate weak antagonism. The effects of the anti-hepatitis B virus (anti-HBV) and anti-HCV agents adefovir and ribavirin on: (1) bictegravir alone; (2) the compound of formula II alone; and the combinations of (1) bictegravir and the compound of formula II; and (2) the bictegravir and the compound of formula III are examined using linear regression, as described previously [41]. Because the HIV-1 IIIB MT-4 system is CXCR4-based, the CCR5 inhibitor, maraviroc, is evaluated in a checkerboard dilution format using MAGI-CCR5 cells with the Gal Screen reagent (Tropix®, Bedford, Mass.) for chemiluminescent endpoints, and data are analyzed as described by Prichard and Shipman [37] by using the MacSynergy II® program. Synergy volumes in the range of −50 to 50 define additivity; <−50, antagonism; and >50, synergy.

### REFERENCES¹

1. Adachi, et al., J. Virol. 59:284-291 (1986).

9. Chackerian, et al., J. Virol. 71:3932-3939 (1997).

12. Daluge, et al., Antimicrob. Agents Chemother. 38:1590-1603. (1994).

15. Garvey, et al. Antimicrob. Agents Chemother. 52:901-908 (2008).

20. Isaka, et al., Virology 264:237-243 (1999).

21. Jármy, et al., J. Med. Virol. 64:223-231 (2001).

37. Prichard, et al., Antiviral Res. 14:181-205 (1990).

39. Selleseth, et al., Antimicrob. Agents Chemother. 47:1468-1471 (2003).

41. Tukey, et al., Biometrics 41:295-301 (1985). ¹ The reference numbering used in this example is the same as that used in Kobayashi, et al., supra.

1. A combination comprising a compound of bictegravir (formula I): 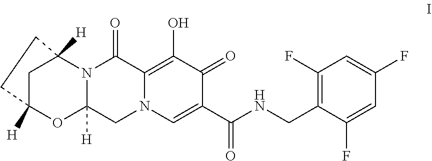 [SMILES: O=C(NCc1c(F)cc(F)cc1F)c1cn2c(c(O)c1=O)C(=O)N1[C@H]3CC[C@H](C3)O[C@@H]1C2]
or a pharmaceutically acceptable salt thereof; and
a compound of formula II: 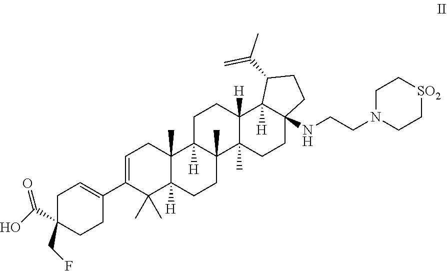
or a pharmaceutically acceptable salt thereof.

2. A pharmaceutical composition comprising the combination as claimed in claim 1 in association with one or more pharmaceutically acceptable carriers therefor.

3-4. (canceled)

5. A method for treating human immunodeficiency virus (HIV) in a human, comprising administering to the human a therapeutically effective amount of bictegravir (formula I): 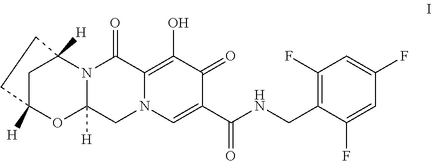 [SMILES: O=C(NCc1c(F)cc(F)cc1F)c1cn2c(c(O)c1=O)C(=O)N1[C@H]3CC[C@H](C3)O[C@@H]1C2]
or a pharmaceutically acceptable salt thereof; and
a therapeutically effective amount of a compound of formula II: 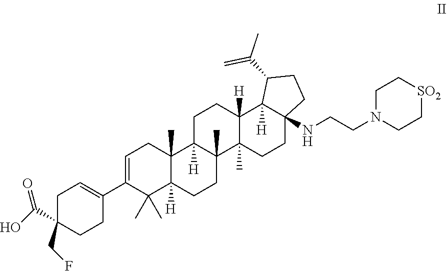
or a pharmaceutically acceptable salt thereof.

6. The method as claimed in claim 5, wherein bictegravir, or a pharmaceutically acceptable salt thereof, and the compound of formula II, or a pharmaceutically acceptable salt thereof, are co-administered in separate dosage forms.

7. The method as claimed in claim 5, wherein bictegravir, or a pharmaceutically acceptable salt thereof, and the compound of formula II, or a pharmaceutically acceptable salt thereof, are co-administered in a single dosage form.

8. The method as claimed in claim 5 wherein the method further comprises administering an agent chosen from: emtricitabine and lamivudine.

9. A method for preventing human immunodeficiency virus (HIV) in a human, comprising administering to the human a therapeutically effective amount of bictegravir (formula I): 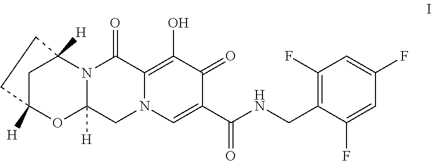 [SMILES: O=C(NCc1c(F)cc(F)cc1F)c1cn2c(c(O)c1=O)C(=O)N1[C@H]3CC[C@H](C3)O[C@@H]1C2]
or a pharmaceutically acceptable salt thereof; and
a therapeutically effective amount of a compound of formula II: 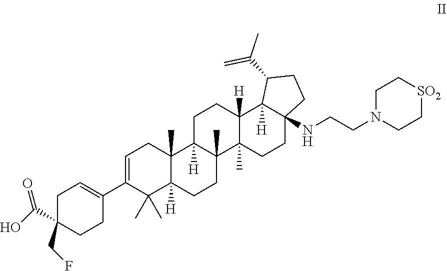
or a pharmaceutically acceptable salt thereof.

10. The method as claimed in claim 9, wherein bictegravir, or a pharmaceutically acceptable salt thereof, and the compound of formula II, or a pharmaceutically acceptable salt thereof, are co-administered in separate dosage forms.

11. The method as claimed in claim 9, wherein bictegravir, or a pharmaceutically acceptable salt thereof, and the compound of formula II, or a pharmaceutically acceptable salt thereof, are co-administered in a single dosage form.

12. The method as claimed in claim 9 wherein the method further comprises administering an agent chosen from: emtricitabine and lamivudine.

13. A pharmaceutical composition comprising a therapeutically effective amount of bictegravir (formula I): 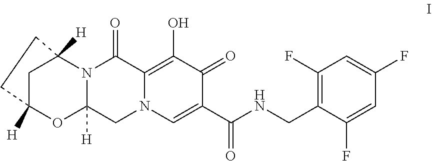 [SMILES: O=C(NCc1c(F)cc(F)cc1F)c1cn2c(c(O)c1=O)C(=O)N1[C@H]3CC[C@H](C3)O[C@@H]1C2]
or a pharmaceutically acceptable salt thereof; and
a therapeutically effective amount of a compound of formula II: 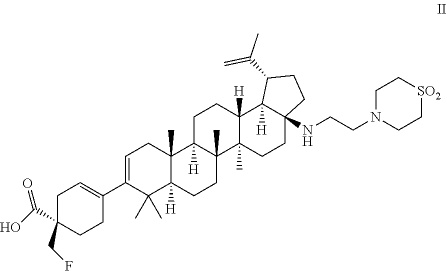
or a pharmaceutically acceptable salt thereof, and at least one pharmaceutically acceptable carrier.

14. The pharmaceutical composition as claimed in claim 13, wherein said composition optionally comprises ritonavir.

15. The pharmaceutical composition as claimed in claim 13 wherein said composition further comprises an agent chosen from: emtricitabine and lamivudine.

16. A kit comprising:
(1) a composition comprising bictegravir;
(2) a composition comprising the compound of formula II; and
(3) instructions for their co-administration.

17-32. (canceled)
